(19) United States
(12) Patent Application Publication
(10) Pub. No.: US 2021/0137958 A1
(43) Pub. Date: May 13, 2021

(54) PHARMACEUTICAL COMPOSITIONS OF DECITABINE

(71) Applicant: SHILPA MEDICARE LIMITED, RAICHUR (IN)

(72) Inventors: PRADEEP SHIVAKUMAR, Bengaluru (IN); PURUSHOTHAMA NARASIYAPPA, Bengaluru (IN); KIRAN KUMAR CHERUKURI, Bengaluru (IN); SREENIVASA REDDY, Bengaluru (IN)

(73) Assignee: SHILPA MEDICARE LTD, RAICHUR (IN)

(21) Appl. No.: 16/618,342

(22) PCT Filed: May 23, 2019
(86) PCT No.: PCT/IB2019/054261

§ 371 Date: Nov. 30, 2019

(30) Foreign Application Priority Data
May 25, 2018 (IN) 201841019570

(51) Int. Cl. A61K 31/706; A61K 47/12; A61K 47/18; A61K 9/00; A61K 9/08

(52) U.S. Cl. CPC A61K 31/706; A61K 47/12; A61K 9/08; A61K 9/0019; A61K 47/186

(57) ABSTRACT

The present invention relates to stable liquid pharmaceutical composition of decitabine comprising decitabine and at least one pharmaceutically acceptable excipient, wherein moisture content of the liquid composition is less than about 2.0% by weight. The invention further relates to stable liquid composition of decitabine wherein the total impurities of the composition is less than about 2.0% by weight when stored at 25° C./60% RH for at least 6 months. The invention further relates to method of using such stable liquid compositions of decitabine for parenteral administration either as ready-to-use or ready-to-dilute for treating various cancer disorders.

### FIELD OF THE INVENTION

The present invention relates to stable liquid compositions of decitabine (5-aza-2′-deoxycytidine) and process to prepare the stable liquid compositions of decitabine. The present invention relates to stable pharmaceutical liquid composition of decitabine comprising decitabine and at least one pharmaceutically acceptable excipient wherein moisture content of the liquid composition is less than about 2.0% by weight. The invention further relates to stable liquid composition of decitabine prepared by a process comprising a step of reducing the moisture content from liquid composition of decitabine. The invention further relates to method of using such stable liquid composition of decitabine for parenteral administration either as ready-to-use or ready-to-dilute for treating various cancer disorders. More specifically the present invention relates to stable, ready-to-dilute pharmaceutical composition comprising decitabine (5-aza-2′-deoxycytidine) and eutectic solvent, wherein moisture content of the pharmaceutical composition is less than about 2.0% by weight. The pharmaceutical compositions may further comprise at least one polar protic solvent and/or at least one polar aprotic solvent. The pharmaceutical compositions thus prepared can be specifically used for the treatment of patients suffering from myelodysplastic syndromes.

### BACKGROUND OF THE INVENTION

Decitabine (5-aza-2′-deoxycitidine) is an analogue of the natural nucleoside 2′-deoxycytidine chemically represented as 4-amino-1-(2-deoxy-β-D-erythro-pentofuranosyl)-1,3,5-triazin-2(1H)-one) and has the structural formula:

[chemical structure]

Decitabine is believed to exert its antineoplastic effects after phosphorylation and direct incorporation into DNA and inhibition of DNA methyltransferase, causing hypomethylation of DNA and cellular differentiation or apoptosis. Decitabine inhibits DNA methylation in vitro, which is achieved at concentrations that do not cause major suppression of DNA synthesis. Decitabine-induced hypomethylation in neoplastic cells may restore normal function to genes that are critical for the control of cellular differentiation and proliferation. In rapidly dividing cells, the cytotoxicity of decitabine may also be attributed to the formation of covalent adducts between DNA methyltransferase and decitabine incorporated into DNA. Non-proliferating cells are relatively insensitive to decitabine.

Decitabine is commercially supplied as a sterile lyophilized powder for injection with the brand name Dacogen® in a clear glass vial and each 20 mL glass vial contains 50 mg decitabine, 68 mg monobasic potassium phosphate (potassium dihydrogen phosphate) and 116 mg sodium hydroxide. As per the package insert leaflet of Dacogen®, it has to be aseptically reconstituted with 10 mL of sterile water for Injection (USP). Upon reconstitution each mL contains approximately 5 mg of Decitabine at pH 6.7-7.3. Immediately after reconstitution, the solution has to be further diluted with 0.9% sodium chloride injection, 5% dextrose injection to a final drug concentration of 0.1 to 1 mg/mL. Unless used within 15 minutes of reconstitution, the diluted solution must be prepared using cold (2° C.-8° C.) infusion fluids and stored at 2° C.-8° C. (36° F.—46°) for up to maximum of 4 hours until administration. It is apparent that decitabine is highly unstable in aqueous media.

Decitabine is most typically administered to patients by injection such as continuous intravenous infusion at a dose of 15 mg/m² over 3 hours repeated every 8 hours for 3 days or continuous intravenous infusion at a dose of 20 mg/m² over one hour repeated daily for 5 days. The length of intravenous infusion is limited by the fast decomposition of decitabine in the aqueous media. The disadvantages of lyophilized drugs are that they have to be reconstituted, usually by injecting a diluent through the septum into the vial. The drug is then drawn up into a new syringe, the needle has to be changed before the drug is finally being injected into the patient. These multiple steps are inconvenient and bear the risk of injuries from the exposed needles. This is especially disadvantages for cytotoxic drugs such as Decitabine.

Decitabine is known to undergo degradation by oxidation and hydrolysis. U.S. Pat. No. 6,982,253 discloses liquid formulations of decitabine solvated in a solvent that comprises glycerin, propylene glycol, polyethylene glycol or combinations thereof and comprises less than 40% water. The liquid formulations further comprise additional excipients such as drying agents, acidifying agents (organic and inorganic acids) and diluents selected from group consisting of mannitol, sorbitol, lactose, dextrose and cyclodextrin. Also, the decitabine used in the preparation of the formulation has a defined particle size of at least 90% of the particles is below 20 μm, and optionally, the size of at least 50% of the particles is below 10 μm, which is achieved by micronization. Further the pharmaceutical compositions according to US '253 are unstable and require micronization of the drug due to its limited solubility in the described solvent systems as well as the higher viscosity of the latter.

U.S. Pat. No. 9,669,048 discloses ready to use pharmaceutical composition comprising unmicronized decitabine, at least one protic solvent, and at least one aprotic solvent, wherein the composition is non-aqueous, that at least one protic solvent is ethanol, the at least one aprotic solvent is dimethylacetamide, and the protic solvent and the aprotic solvent are present in the ratio of 10:90 to 50:50 (protic:aprotic). The pharmaceutical composition according to US '048 compositions are unstable.

However, none of the above references disclose a stable formulations or compositions of decitabine which are stable for longer period of time. Hence there exists is a need for stable ready-to-dilute or ready-to-use pharmaceutical compositions of decitabine overcoming the disadvantages of the prior art.

The first object of the present invention is to provide stable pharmaceutical liquid composition of decitabine comprising decitabine and at least one pharmaceutically acceptable excipient wherein moisture content of the liquid composition is less than about 2.0% by weight, preferably 1.0% by weight and more preferably 0.5% by weight.

Second object of the present invention is to provide ready-to-dilute or ready-to-use pharmaceutical compositions comprising decitabine (5-aza-2′-deoxycytidine); eutectic solvent consisting of choline chloride and urea; and at least one protic solvent. The pharmaceutical compositions may further comprise at least one pharmaceutically acceptable aprotic solvent.

Another object of the present invention is to provide ready-to-dilute or ready-to-use pharmaceutical compositions comprising decitabine (5-aza-2′-deoxycytidine); eutectic solvent consisting of choline chloride and urea; and at least one protic solvent, wherein moisture content of the pharmaceutical composition is less than about 2.0% by weight.

It is yet another object of the present invention that includes stable liquid compositions of decitabine wherein the moisture content from stable liquid compositions of decitabine is further reduced by means of adsorbents selected from the group consisting of molecular sieves, silica gel, activated alumina, activated charcoal or mixtures thereof.

Another object of the present invention to control the oxygen content by the additions of antioxidants or by using an inert gas such as nitrogen.

Another object of the present invention is to provide pharmaceutical compositions that are stable at 2° C.-25° C. storage conditions.

Yet another object of the present invention is to avoid the micronization of decitabine.

Yet another object of the invention is to provide more cost efficient and economical formulations of decitabine.

### SUMMARY OF THE INVENTION

The present invention relates to stable liquid compositions of decitabine and process to prepare such stable liquid compositions of decitabine. The invention relates to stable pharmaceutical liquid compositions of decitabine comprising decitabine and at least one pharmaceutically acceptable excipient wherein moisture content of the liquid compositions is less than about 2.0% by weight. The invention also relates to stable liquid compositions of decitabine prepared by a process comprising a step of reducing the moisture content from liquid compositions of decitabine. The invention further relates to method of using such stable liquid compositions of decitabine for treating various cancer disorders.

In one embodiment the invention includes stable liquid compositions of decitabine comprising decitabine and at least one pharmaceutically acceptable excipient.

In another embodiment the invention relates to stable liquid compositions of decitabine comprising decitabine in pharmaceutically effective concentration and at least one pharmaceutically acceptable excipient.

In embodiments of the invention pharmaceutically acceptable excipients include solvents and antioxidants. Solvents are selected from the group consisting of eutectic solvents, polar protic solvents and polar aprotic solvents.

In an embodiment the invention provides stable liquid compositions of decitabine wherein pharmaceutically effective concentration of decitabine is at least 0.01 mg/mL.

In another embodiment the present invention relates to stable liquid compositions of decitabine comprising decitabine and eutectic solvent comprising halide salt and a hydrogen bond donor. The liquid compositions may further comprise at least one polar protic solvent or polar aprotic solvent.

In a further embodiment the present invention relates to stable liquid compositions of decitabine comprising a) decitabine, b) eutectic solvent consisting halide salt and a hydrogen bond donor, and c) at least one polar protic solvent.

In another embodiment of the invention, the present invention relates to stable liquid compositions of decitabine comprising a) decitabine, b) eutectic solvent comprising halide salt and a hydrogen bond donor, c) at least one polar protic solvent and d) at least one antioxidant.

In a further embodiment of the present invention, the present invention relates to a stable liquid composition of decitabine comprising decitabine and eutectic solvent consisting of urea and choline chloride.

In another embodiment of the present invention, the present invention provides a stable liquid composition of decitabine comprising a) decitabine, b) eutectic solvent consisting of urea and choline chloride and c) at least one polar protic solvent.

In another embodiment the invention includes stable liquid compositions of decitabine prepared by a process comprising a step of reducing the moisture content from liquid compositions of decitabine.

Further embodiment includes stable liquid composition of decitabine wherein liquid composition are prepared by a process comprising the steps of

a) preparing a bulk solution of decitabine by dissolving decitabine in a suitable solvent,
b) incubating the bulk solution of decitabine solution of step a) with adsorbents or mixture of adsorbents in suitable ratio for suitable period of time, and
c) optionally filtering the decitabine solution from step b) by using suitable filter followed by filling into vials, stoppering and sealing.

In another embodiment the invention includes stable liquid compositions of decitabine wherein the liquid compositions are prepared by a process comprising a step of reducing the moisture content from the liquid compositions of decitabine, wherein the process comprises the steps of

a) preparing the solution of decitabine by dissolving decitabine in a suitable solvent,
b) preparing at least one column by using suitable adsorbent or mixture of adsorbents,
c) passing the decitabine solution of step a) through the column or series of columns of step b) and
d) optionally filtering the solution through suitable filter followed by filling into vials, stoppering and sealing.

In one embodiment of the present invention the oxygen content is controlled by the addition of the antioxidants and by using an inert gas such as nitrogen.

In another embodiment of the present invention, decitabine is used in unmicronized form for dissolving in the suitable solvent.

In yet another embodiment of the present invention the pharmaceutical compositions are stable 2° C.-25° C. storage conditions.

In yet another embodiment of the inventions the compositions are cost efficient and economical.

### DETAILED DESCRIPTION OF THE INVENTION

In first aspect the present invention relates to stable liquid compositions of decitabine comprising decitabine and at least one pharmaceutically acceptable excipient wherein moisture content of the liquid compositions is less than 5.0% by weight.

In embodiments of the invention pharmaceutically acceptable excipients are selected from the group comprising of solvents and antioxidants.

In another embodiment solvents are selected from group consisting of eutectic solvents, polar protic solvents and polar aprotic solvents.

The term “stable” or “stability” as used herein relate to both physical and chemical stability, wherein pharmaceutical composition of decitabine can be stored for commercially significant periods, such as at least 3 moths, 6 months, one year or two years or 3 years without significant physical instability (description and clarity etc) and chemical degradation. The stable may represent stability when stored at 2° C.-8° C., or at ambient conditions (e.g 25° C.) or elevated temperatures (e.g 40° C.). Percent degradation may be determined by analysing impurities by suitable analytical method.

The term “pharmaceutically acceptable” refer to ingredients that are useful for preparing pharmaceutical compositions that are acceptable for human pharmaceutical use.

The term “substantial” or “substantially” as used to describe percentage of decitabine in the formulation is at least 90%, or at least about 95%, or at least of 99%.

The term “moisture content” or water content or bound water refer to the water content in the composition or formulation. The moisture content may be bound or in unbound form.

The term “ready-to-dilute” or RTD composition is a stable liquid composition of decitabine that is diluted with suitable diluent for parenteral administration. Suitable diluents may include sterile water for injection, 0.9% sodium chloride, 0.45% sodium chloride, 5% dextrose or combinations thereof.

The term “ready-to-use” or RTU composition is a stable liquid composition of decitabine which are ready for administration in the form of parenteral administration.

The term “incubate” or “incubation” denotes the decitabine solution is in contact with the adsorbent for certain period of time or adsorbents are immersed in bulk solution of decitabine for certain period of time.

The term “adsorbent” includes any substance used to remove or reduce moisture or water content from decitabine or its compositions. Substance may be excipients or mixtures thereof. Sometimes adsorbents also refer to desiccants.

The term “pharmaceutically effective concentration” refers to any concentration of decitabine showing its therapeutic effect.

The bulk solution of drug or decitabine as discussed herein this invention refers to any solution prepared by dissolving drug or decitabine in a suitable solvent optionally with stirring.

The term “composition” in the present invention refers to combination of drug or decitabine along with at least one pharmaceutically acceptable excipient and used in preparing pharmaceutical formulations with no specific limitations.

The term “formulation” refers to pharmaceutical dosage forms containing compositions of decitabine. The pharmaceutical formulations of the present invention can be prepared as solutions or suspensions or dispersions and so on presented in glass ampoules or glass vials or any suitable devices.

In an embodiment of the invention the invention includes the stable liquid compositions of decitabine comprising decitabine and pharmaceutically acceptable excipients with moisture content of less than about 2.0% by weight of composition.

In an embodiment of the invention the invention includes the stable liquid compositions of decitabine comprising decitabine and pharmaceutically acceptable excipients with the moisture content of less than about 2.0% by weight of composition, preferably 1.5% by weight, more preferably 1.0% by weight and most preferably 0.5% by weight, when stored at 2° C.-8° C. for at least 6 months.

In further embodiments of the invention the invention includes the stable liquid compositions of decitabine comprising decitabine and pharmaceutically acceptable excipients with the moisture content of less than about 2.0% by weight, preferably 1.5% by weight, more preferably 1.0% by weight and most preferably 0.5% by weight, of composition when stored at 25° C./60% RH for at least 6 months.

In an embodiment of the invention the present invention provides the stable liquid compositions of decitabine wherein the pharmaceutically effective concentration is at least 0.01 mg/mL.

In second aspect the present invention relates to ready-to-dilute or ready-to-use pharmaceutical compositions comprising decitabine and eutectic solvent.

In embodiments of the invention, the ready-to-dilute or ready-to-use pharmaceutical compositions of decitabine comprise the eutectic solvent manufactured by halide salt and hydrogen bond donor as mentioned below table in the stoichiometric ratio of 0.25:10 to about 10.0:0.25; more preferably in the ratio of about 0.25:5.0 to about 5.0:0.25; and most preferably 0.5:3.0 to about 3.0:0.5.

TABLE 1
Eutectic mixtures solvents

| Halide Salt | Hydrogen Bond Donor |
|---|---|
| Choline chloride | Urea |
| Choline chloride | Thiourea |
| Choline chloride | 1-methyl urea |
| Choline chloride | 1,1-dimethyl urea |
| Choline chloride | 1,3-dimethyl urea |
| Choline chloride | 2,3-xylenol |
| Choline chloride | Acetamide |
| Choline chloride | Banzamide |
| Choline chloride | Ethylene glycol |
| Choline chloride | Glycerol |
| Choline chloride | Adipic acid |
| Choline chloride | Benzoic acid |
| Choline chloride | Citric acid |
| Choline chloride | Imidazole |
| Choline chloride | Itaconic acid |
| Choline chloride | Malonic acid |
| Choline chloride | O-cresol |
| Choline chloride | Oxalic acid |
| Choline chloride | P-coumaric acid |
| Choline chloride | Phenol |
| Choline chloride | Phenylacetic acid |
| Choline chloride | Phenylpropionic acid |
| Choline chloride | Suberic acid |
| Choline chloride | Succinic acid |
| Choline chloride | Tricarballylic acid |
| Choline chloride | MgCl₂•6H₂O |
| Choline chloride | Tartaric acid |
| Choline chloride | Trans-cinnamic acid |

In embodiments of the invention the present invention relates to stable liquid compositions of decitabine comprising decitabine and eutectic solvent consisting of halide salt and a hydrogen bond donor.

In embodiments of the invention preferably used eutectic solvent is the mixture of choline chloride and urea.

In further embodiment of the invention the present invention relates to stable liquid compositions of decitabine comprising decitabine and eutectic solvent consisting of choline chloride and urea.

In embodiments of the invention the pharmaceutical compositions of present invention further comprise at least one polar protic solvent or at least one polar aprotic solvent.

In embodiments of the invention polar protic solvents are selected from the group consisting of n-butanol, isopropanol, ethanol and methanol or mixtures thereof. The most preferably used polar protic solvent is ethanol (dehydrated alcohol, absolute alcohol or anhydrous alcohol).

In embodiments of the present invention, the present invention relates to stable liquid composition of decitabine comprising a) decitabine, b) eutectic solvent consisting of urea and choline chloride and c) ethanol.

In embodiments of the invention polar aprotic solvents are selected from the group consisting of 1-methyl-2-pyrrolidone, 1,3-dimethyl-2-imidazolidinone, dimethylacetamide, dimethyl sulfoxide, acetone, tetrahydrofuran, 1,4-dioxane, acetonitrile, dimethyl formamide, propylene carbonate, or mixtures thereof. The most preferably used polar aprotic solvents are dimethylacetamide, dimethyl sulfoxide, or mixtures thereof.

In embodiments of the present invention, the oxygen content is controlled by using antioxidants and/or inert gas such as nitrogen. In the embodiments of the invention, the present invention further comprises the antioxidants. Antioxidants are selected from the group consisting of formic acid, sodium metabisulphite, acetone sodium metabisulphite, sodium formaldehyde sulfoxylate, citric acid, d,l-α-tocopherol, butylated hydroxy anisole, butylated hydroxy toluene, monothioglycerol, ascorbic acid, propyl gallate or the like. The most preferably used antioxidant is formic acid.

Further the oxygen content may be also aided by, purging the sealable container with a gas which is substantially oxygen-free, or substantially moisture free, or substantially oxygen and moisture free. Purging can be expected to reduce the oxygen level in the sealable container to a level of from about 0.5% to about 10%, typically about 5% or lower, depending on the efficiency of flushing and how quickly the container is sealed after flushing. Oxygen level in the sealable container is from about 0.1 ppm to about 10 ppm, preferably less than 5 ppm and most preferably less than 2 ppm. The gas used for purging the sealable container may be any appropriate inert gas known to those in the art, the most commonly used gases being argon, helium or nitrogen, or mixtures thereof. However, the most preferred inert gas is nitrogen.

In embodiments of the invention the present invention relates to stable liquid composition of decitabine consisting essentially of a) decitabine, b) eutectic solvent consisting of urea and choline chloride, c) at least one polar protic solvent and d) at least one antioxidant.

In embodiments of the invention includes stable liquid compositions of decitabine prepared by a process comprising a step of further reducing the moisture content from liquid compositions of decitabine.

As discussed above, decitabine is highly unstable in presence of moisture/water content or temperature. From the forced degradation data of decitabine, it has been observed that four known impurities namely Impurity-1, Impurity-2, Impurity-3 and NFDU are obtained under different stressed conditions as shown below.

a) Impurity 1 is formed in acidic and basic conditions. Impurity 1 is chemically 5-Azacytosine or 4-amino-1, 3, 5-triazin-2(1H)-one and represented by following structure.

[chemical structure]

b) Impurity 2 is an oxidation impurity formed by peroxide stress and is chemically (4-amino-1-(tetrahydro-4-hydroxy-5-(Hydroxymethyl)furan-2-yl)-1,3,5-triazin-2(1H)-one or alpha isomer and represented by following structure

[chemical structure]

c) Impurity 3 is an oxidation impurity formed by peroxide stress. Impurity 3 is chemically represented as (2-Deoxy-ribufuranosyl-3-guanylurea) or deformyl impurity and represented by following structure

[chemical structure]

d) NFDU impurity is a transition impurity and increases on oxidation by peroxide stress chemically represented as N-(formylamidino) N′-β-D-2′-deoxyribofuranosylurea and structurally represented as

[chemical structure]

In an embodiment of the invention relates to stable liquid compositions of decitabine wherein levels of impurity 2 and NFDU are less than about 2.0%, more preferably less than about 1.0% when stored at 2° C.-8° C. for at least 6 months.

In further embodiment of the invention relates to stable liquid compositions of decitabine wherein levels of impurity 2 and NFDU are less than about 2.0%, more preferably less than about 1.0% when stored at 25° C./60% RH for at least 6 months.

In another embodiment of the invention relates to stable liquid compositions of decitabine wherein total decitabine related impurities is less than about 5.0%, preferably less than 3.0% and more preferably less than 2.0% when stored at 2° C.-8° C. for at least 6 months.

In a further embodiment of the invention relates to stable liquid compositions of decitabine wherein total decitabine related impurities is less than about 5.0%, preferably less than 3.0% and more preferably less than 2.0% when stored at 25° C./60% RH for at least 6 months.

In an embodiment of the invention includes stable liquid compositions of decitabine wherein the moisture content from liquid compositions of decitabine is reduced by means of adsorbents selected from the group comprising molecular sieves, silica gel, activated alumina and activated charcoal.

Molecular sieves are pelleted, beaded and powdered material made from three dimensional materials. A molecular sieve is a material with very small holes of precise and uniform size. These holes are small enough to block large molecules while allowing small molecules to pass. The small molecules are efficient to pass through the pores and when activated they become powerful adsorbents in a wide range of operating conditions with a strong absorption ability with water, hydrogen, oxygen, carbon dioxide and other polar molecules. The term “activated” with respect to adsorbents refers to the process wherein the molecular sieves are heated to certain temperature for certain period of time. For example, molecular sieves when heated at 120° C. for about 12 hours may be referred as activated molecular sieves.

Molecular sieves are used as adsorbents for gases and liquids. Molecules small enough to pass through the pores are adsorbed while larger molecules are not.

It is different from a common filter in that it operates on a molecular level and traps the adsorbed substance. For instance, a water molecule may be small enough to pass through the pores while larger molecules are not, so water is forced into the pores which act as a trap for the penetrating water molecules, which re retained within the pores. Because of this, they often function as a desiccant. A molecular sieve can adsorb water up to 22% of it owns weight. The principle of adsorption to molecular sieve particles in somewhat similar to that of size exclusion chromatography, except that without a changing solution composition, that absorbed products remained trapped because, in the absence of other molecule able to penetrate the pore and fill the space, a vacuum would be created by desorption.

There are different types of molecular sieves available. Molecular sieves can be microporous, mesoporous or macroporous material.

Microporous (<2 nm) eg: Zeolites (aluminosilicate minerals; porous glass, active carbon, clays such as montmorillonite, halloysite etc.

Mesoporous material (2-50 nm) eg: silicon dioxide (used to make silica gel).

Macroporous material (>50 nm) eg; mesoporous silica.

Further there are different models available for molecular sieves based on their adsorption capabilities including pore diameter, bulk density, adsorbed water etc.

For example:

13× and 10× molecular sieves (beads, pellets and powder)

3A molecular sieves (beads, pellets and powder)

4A molecular sieves (beads, pellets and powder)

5A molecular sieves (beads, pellets and powder)

Among the available models of molecular sieves type 3A molecular sieve was used for removing or reducing water from liquid compositions considering it unique pore size.

By using molecular sieves the water can be removed or reduced by different methods such as static process, wherein adsorbent or mixture of adsorbents in certain weight ratio were incubated or immersed in decitabine bulk solution; and dynamic process wherein the bulk solution of decitabine was passed through the columns containing adsorbents or mixture of adsorbents. The dynamic process can further be in a recirculation method wherein the bulk solution was continuously recirculated through the column using peristaltic pump. Recirculation will increase the efficiency of absorption.

In embodiments of the invention includes the stable liquid compositions of decitabine wherein the ratio of decitabine bulk solution to adsorbent is 1:0.05 to about 1:50, preferably 1:0.05 to 1:25, even more preferably 1:0.1 to 1:10 and most preferably 1:0.1 to 1:2.

In embodiments of the invention the invention includes stable liquid compositions of decitabine wherein the liquid compositions are prepared by a process comprising the steps of:

a) preparing a bulk solution of decitabine by dissolving decitabine in a suitable solvent,

b) incubating the bulk solution of decitabine of step a) with adsorbents or mixture of adsorbents in suitable ratio for suitable period of time, and

c) optionally filtering the decitabine solution from step b) by using suitable filter followed by filling into vials, stoppering and sealing.

In embodiments of the invention the invention includes stable liquid compositions of decitabine herein the liquid compositions are prepared by a process comprising the steps of

a) preparing a bulk solution of decitabine comprising decitabine, eutectic solvent consisting of choline chloride and urea, a polar protic solvent and antioxidant,

b) incubating the bulk solution of decitabine of step a) with adsorbents or mixture of adsorbents in suitable ratio for suitable period of time, and

c) optionally filtering the decitabine solution from step b) by using suitable filter followed by filling into vials, stoppering and sealing.

In further embodiments of the invention the invention includes stable liquid compositions of decitabine herein the liquid compositions are prepared by a process comprising the steps of

a) preparing a bulk solution of decitabine comprising decitabine, eutectic solvent consisting of choline chloride and urea, ethanol and formic acid,

b) incubating the bulk solution of decitabine of step a) with adsorbents or mixture of adsorbents in suitable ratio for suitable period of time, and

c) optionally filtering the decitabine solution from step b) by using suitable filter followed by filling into vials, stoppering and sealing.

In another embodiment the invention includes stable liquid compositions of decitabine wherein the liquid compositions are prepared by a process comprising a step of reducing the moisture content from the liquid compositions of decitabine, wherein the process comprises the steps of

a) preparing the solution of decitabine by dissolving decitabine in a suitable solvent,
b) preparing at least one column by using suitable adsorbent or mixture of adsorbents,
c) passing the decitabine solution of step a) through the column or series of columns of step b) and
d) optionally filtering the solution through suitable filter followed by filling into vials, stoppering and sealing.

In a further embodiment the invention includes stable liquid compositions of decitabine wherein the liquid compositions are prepared by a process comprising a step of reducing the moisture content from the liquid compositions of decitabine, wherein the process comprises the steps of

a) preparing a bulk solution of decitabine comprising decitabine, eutectic solvent consisting of choline chloride and urea, a polar protic solvent and antioxidant,
b) preparing at least one column by using suitable adsorbent or mixture of adsorbents,
c) passing the decitabine solution of step a) through the column or series of columns of step b) and
d) optionally filtering the solution through suitable filter followed by filling into vials, stoppering and sealing.

In a still further embodiment the invention includes stable liquid compositions of decitabine wherein the liquid compositions are prepared by a process comprising a step of reducing the moisture content from the liquid compositions of decitabine, wherein the process comprises the steps of

a) preparing a bulk solution of decitabine comprising decitabine, eutectic solvent consisting of choline chloride and urea, ethanol and formic acid,
b) preparing at least one column by using suitable adsorbent or mixture of adsorbents,
c) passing the decitabine solution of step a) through the column or series of columns of step b) and
d) optionally filtering the solution through suitable filter followed by filling into vials, stoppering and sealing.

In preferred embodiment the formulations are present as a single vial presentation comprising decitabine in a concentration of about 0.01 mg/ml to about 50 mg/mL. The ready-to-use concentration are present in range of about 0.1 mg/mL to about 1 mg/mL and ready-to-dilute are present in range of about 1 mg/mL to about 50 mg/mL, preferably in range of about 5 mg/mL to about 10 mg/mL and more preferably 10 mg/mL.

In a more preferred embodiment 10 mg/mL ready-to-dilute formulations comprising decitabine is reconstituted with 5 mL of sterile water for Injection and further diluted with 0.9% sodium chloride injection or 5% dextrose injection to a final drug concentration of 0.1 to 1 mg/mL, which is intravenously administered as an infusion to a patient.

The pharmaceutical compositions of the present invention are suitable for parenteral administration. These pharmaceutical compositions are then administered via intravenous infusion to treat patients suffering from myelodysplastic syndromes (MDS) including previously treated and untreated, de novo and secondary MDS of all French-American-British subtypes (refractory anaemia, refractory anaemia with ringed sideroblasts, refractory anaemia with excess blasts, refractory anaemia with excess blasts in transformation, and chronic myelomonocytic leukemia) and intermediate-1, intermediate2, and high-risk International Prognostic Scoring System groups.

In yet another embodiment of the present invention Decitabine is not micronized. It has surprisingly been found that the micronization step is not necessary when preparing the compositions according to the present invention. Hence, these are more cost efficient and economical.

The invention is further illustrated by the following examples, which are not construed to be limiting the scope of the invention.

### EXAMPLES

### Example 1

The composition of the present invention contains Decitabine in a concentration of 10 mg/mL (Ready to dilute injection) in a eutectic mixture solvent. The details of the compositions are shown in Table 2.

TABLE 2
Compositions according to Example 1.

| S. No | Ingredients | Quantity (mg/mL) | |
|---|---|---|---|
| 1 | Decitabine | 10 | mg |
| 2 | Eutectic Mixture Solvent | Qs to 1 | mL |

| | | |
|---|---|---|
| 3 | Nitrogen | qs |

The pharmaceutical composition according to example 1 was prepared by the following process: Eutectic mixture solvent is manufactured using a halide salt and hydrogen bond donor listed in Table 1 in specific stoichiometric ratio. A suitable quantity of eutectic mixture solvent was fed into the product manufacturing vessel. Decitabine was added and stirred in about 80% of solution obtained in the previous step and dissolved. The volume was then made up to 100% with the above eutectic mixture solvent. The resulting Decitabine solution was then filtered through a suitable sterilizing grade filter and filled into vials. The vial headspace was then blanketed with nitrogen to achieve a headspace oxygen content of less than 10%, preferably less than 5%. Finally, the vials were stoppered and sealed.

### Example 2

The composition of the present invention contains Decitabine in a concentration of 10 mg/mL (Ready to dilute injection) in a eutectic mixture solvent. The details of the compositions are shown in Table 3.

TABLE 3
Compositions according to Example 2.

| S. No | Ingredients | Quantity (mg/mL) | |
|---|---|---|---|
| 1 | Decitabine | 10 | mg |
| 2 | Eutectic Mixture Solvent | Qs to 1 | mL |

| | | |
|---|---|---|
| 3 | Antioxidant | Qs to water content in formulation |
| 4 | Nitrogen | qs |

The pharmaceutical composition according to example 2 was prepared by the following process: Eutectic mixture solvent is manufactured using a halide salt and hydrogen bond donor listed in Table 1 in specific stoichiometric ratio. Add required quantity of Formic acid equivalent to water content in the Eutectic mixture solvent. A suitable quantity of eutectic mixture solvent was fed into the product manufacturing vessel. Decitabine was added and stirred in about 80% of solution obtained in the previous step and dissolved. Add required quantity of antioxidant equivalent to water content in Decitabine. The volume was then made up to 100% with the above eutectic mixture solvent. The resulting Decitabine solution was then filtered through a suitable sterilizing grade filter and filled into vials. The vial headspace was then blanketed with nitrogen to achieve a headspace oxygen content of less than 10 ppm, preferably less than 2 ppm. Finally, the vials were stoppered and sealed.

### Example 3

The composition of the present invention contains Decitabine in a concentration of 10 mg/mL (Ready to dilute injection) in a eutectic mixture solvent and protic or aprotic solvent. The details of the compositions are shown in Table 4.

TABLE 4
Compositions according to Example 3.

| S. No | Ingredients | Quantity (mg/mL) | |
|---|---|---|---|
| 1 | Decitabine | 10 | mg |
| 2 | Eutectic Mixture Solvent | 0.5 | mL |
| 3 | Protic or aprotic solvent | Qs to 1 | mL |

| | | |
|---|---|---|
| 4 | Nitrogen | qs |

The pharmaceutical composition according to example 3 was prepared by the following process:

Eutectic mixture solvent is manufactured using a halide salt and hydrogen bond donor listed in Table 1 in specific stoichiometric ratio. Add required quantity of Formic acid equivalent to water content in the Eutectic mixture solvent. A suitable quantity of eutectic mixture solvent was fed into the product manufacturing vessel. Decitabine was added and stirred in about 80% of solution obtained in the previous step and dissolved. Add required quantity of Formic acid equivalent to water content in Decitabine. The volume was then made up to 100% with Protic or aprotic solvent. The resulting Decitabine solution was then filtered through a suitable sterilizing grade filter and filled into vials. The vial headspace was then blanketed with nitrogen to achieve a headspace oxygen content of less than 10 ppm, preferably less than 2 ppm. Finally, the vials were stoppered and sealed.

### Example 4

The composition of the present invention contains Decitabine in a concentration of 10 mg/mL (Ready to dilute injection) in a eutectic mixture solvent listed in Table 1 and protic or aprotic solvent. The details of the compositions are shown in Table 5.

TABLE 5
Compositions according to example 4.

| S. No | Ingredients | Quantity (mg/mL) | |
|---|---|---|---|
| 1 | Decitabine | 10 | mg |
| 2 | Eutectic Mixture Solvent | 0.5 | mL |
| 3 | Protic or aprotic solvent | Qs to 1 | mL |

| | | |
|---|---|---|
| 4 | Antioxidant | Qs to water content in formulation |
| 5 | Nitrogen | Qs |

The pharmaceutical composition according to example 4 was prepared by the following process:

Eutectic mixture solvent is manufactured using a halide salt and hydrogen bond donor listed in Table 1 in specific stoichiometric ratio. A suitable quantity of eutectic mixture solvent was fed into the product manufacturing vessel. Decitabine was added and stirred in about 80% of solution obtained in the previous step and dissolved. The volume was then made up to 100% with protic or aprotic solvent. The resulting Decitabine solution was then filtered through a suitable sterilizing grade filter and filled into vials. The vial headspace was then blanketed with nitrogen to achieve a headspace oxygen content of less than 10 ppm, preferably less than 2 ppm. Finally, the vials were stoppered and sealed.

Example 6 to 10:

| | | Ex: 6 | | Ex: 7 | | Ex: 8 | | Ex: 9 | | Ex: 10 | |
|---|---|---|---|---|---|---|---|---|---|---|---|
| | | Quantity | | Quantity | | Quantity | | Quantity | | Quantity | |
| S. No | Ingredients | (mg/mL) | | (mg/mL) | | (mg/mL) | | (mg/mL) | | (mg/mL) | |
| 1 | Decitabine | 10 | mg | 10 | mg | 10 | mg | 10 | mg | 10 | mg |
| 2 | Choline | 340 | mg | 340 | mg | 340 | mg | 340 | mg | 340 | mg |
| | Chloride | | | | | | | | | | |
| 3 | Urea | 85 | mg | 170 | mg | 255 | mg | 340 | mg | 680 | mg |
| 4 | Formic acid | 0.005 | mg | 0.005 | mg | 0.0005 | mg | 0.0005 | mg | 0.0005 | mg |
| 5 | Ethanol | Q.s to 1 | ml | Qs to 1 | ml | Qs to 1 | ml | Qs to 1 | ml | Qs to 1 | ml |

Manufacturing Process:

Eutectic mixture solvent is manufactured using choline chloride and Urea. Eutectic mixture solvent was fed into the product manufacturing vessel and decitabine was added to eutectic mixture solvent and dissolved. Formic acid was added to the above solution followed by addition of ethanol and stirred to get homogenous solution. The resulting decitabine solution was then filtered through a suitable sterilizing grade filter and filled into vials. The vial headspace was then blanketed with nitrogen to achieve a headspace oxygen content of less than 10 ppm, preferably less than 2 ppm. Finally, the vials were stoppered and sealed. Decitabine formulation prepared according to the invention was tested for stability at 2° C.-8° C. and at 25° C./60% RH for a period of 6 months. Stability of examples 6, 7, 8, 9 and 10 are shown in Tables 6, 7, 8, 9 and 10 respectively.

TABLE 6
Stability data for product as obtained in Example - 6

| Condition/ | | 2° C.-8° C. | | | 25° C./60% RH | |
|---|---|---|---|---|---|---|
| Time Point | Initial | 1M | 3M | 6M | 1M | 3M |
| Moisture Content | 0.5 | 1.2 | 2.5 | 5.5 | 3.2 | 6.7 |
| (%) | | | | | | |
| Impurity - 1 (%) | ND | ND | 0.24 | 0.85 | 0.65 | 0.72 |
| Impurity -2 (%) | ND | 0.56 | 1.23 | 2.1 | 1.6 | 3.4 |
| Impurity -3 (%) | ND | 0.04 | 0.46 | 0.69 | 0.32 | 0.72 |
| NFDU (%) | 0.30 | 0.9 | 1.52 | 2.7 | 1.82 | 3.2 |
| Total Impurities | 0.52 | 2.52 | 4.9 | 8.2 | 5.3 | 10.5 |
| (%) | | | | | | |
| Assay (%) | 99.9 | 98.5 | 95.3 | 90.9 | 94.2 | 88.2 |

*ND—Not detectable

TABLE 7
Stability data for product as obtained in Example - 7

| Condition/ | | 2° C.-8° C. | | | 25° C./60% RH | |
|---|---|---|---|---|---|---|
| Time Point | Initial | 1M | 3M | 6M | 1M | 3M |
| Moisture Content | 0.3 | 0.5 | 0.7 | 1.8 | 0.7 | 1.2 |
| (%) | | | | | | |
| Impurity - 1 (%) | ND | ND | ND | ND | ND | 0.05 |
| Impurity -2 (%) | ND | ND | 0.05 | 0.45 | 0.04 | 0.23 |
| Impurity -3 (%) | ND | ND | ND | ND | ND | ND |
| NFDU (%) | 0.20 | 0.36 | 0.56 | 0.72 | 0.49 | 0.78 |
| Total Impurities | 0.45 | 0.72 | 0.9 | 1.68 | 0.92 | 1.72 |
| (%) | | | | | | |
| Assay (%) | 99.7 | 99.5 | 99.2 | 98.5 | 98.7 | 98.5 |

*ND—Not detectable

TABLE 8
Stability data for product as obtained in Example - 8

| Condition/ | | 2° C.-8° C. | | | 25° C./60% RH | | |
|---|---|---|---|---|---|---|---|
| Time Point | Initial | 1M | 3M | 6M | 1M | 3M | 6M |
| Moisture Content | 0.2 | 0.3 | 0.45 | 1.2 | 0.5 | 0.6 | 0.8 |
| (%) | | | | | | | |
| Impurity - 1 (%) | ND | ND | ND | ND | ND | ND | ND |
| Impurity -2 (%) | ND | ND | ND | 0.12 | ND | ND | 0.43 |
| Impurity -3 (%) | ND | ND | ND | ND | ND | ND | ND |
| NFDU (%) | 0.15 | 0.32 | 0.38 | 0.46 | 0.35 | 0.46 | 0.52 |
| Total Impurities | 0.35 | 0.56 | 0.69 | 0.79 | 0.59 | 0.82 | 1.5 |
| (%) | | | | | | | |
| Assay (%) | 99.8 | 99.6 | 99.4 | 99.2 | 99.1 | 98.7 | 98.3 |

*ND—Not detectable

TABLE 9
Stability data for product as obtained in Example - 9

| Condition/ | | 2° C.-8° C. | | | 25° C./60% RH | |
|---|---|---|---|---|---|---|
| Time Point | Initial | 1M | 3M | 6M | 1M | 3M |
| Moisture Content | 0.4 | 0.6 | 0.8 | 1.5 | 0.9 | 1.9 |
| (%) | | | | | | |
| Impurity - 1 (%) | ND | ND | ND | ND | ND | ND |
| Impurity -2 (%) | ND | ND | 0.03 | 0.16 | 0.09 | 0.4 |
| Impurity -3 (%) | ND | ND | ND | ND | ND | ND |
| NFDU (%) | 0.25 | 0.43 | 0.53 | 0.62 | 0.64 | 0.92 |
| Total Impurities | 0.56 | 0.67 | 0.78 | 1.2 | 1.4 | 1.8 |
| (%) | | | | | | |
| Assay (%) | 100.2 | 99.4 | 99.1 | 98.6 | 98.7 | 98.1 |

*ND—Not detectable

TABLE 10
Stability data for product as obtained in Example - 10

| Condition/ | | 2° C.-8° C. | | | 25° C./60% RH | |
|---|---|---|---|---|---|---|
| Time Point | Initial | 1M | 3M | 6M | 1M | 3M |
| Moisture Content | 0.5 | 0.6 | 0.75 | 1.6 | 1.0 | 1.9 |
| (%) | | | | | | |
| Impurity - 1 (%) | ND | ND | ND | ND | ND | 0.07 |
| Impurity -2 (%) | ND | ND | 0.12 | 0.29 | 0.09 | 0.25 |
| Impurity -3 (%) | ND | ND | ND | ND | ND | ND |
| NFDU (%) | 0.34 | 0.39 | 0.63 | 0.95 | 0.89 | 1.2 |
| Total Impurities | 0.48 | 0.92 | 1.45 | 1.85 | 1.4 | 1.9 |
| (%) | | | | | | |
| Assay (%) | 100.4 | 99.4 | 99.2 | 98.5 | 98.6 | 98.2 |

*ND—Not detectable

### Examples 11 to 15

| | | Ex: 11 | | Ex: 12 | | Ex: 13 | | Ex: 14 | | Ex: 15 | |
|---|---|---|---|---|---|---|---|---|---|---|---|
| | | Quantity | | Quantity | | Quantity | | Quantity | | Quantity | |
| S. No | Ingredients | (mg/mL) | | (mg/mL) | | (mg/mL) | | (mg/mL) | | (mg/mL) | |
| 1 | Decitabine | 10 | mg | 10 | mg | 10 | mg | 10 | mg | 10 | mg |
| 2 | Choline | 340 | mg | 170 | mg | 255 | mg | 680 | mg | 1020 | mg |
| | Chloride | | | | | | | | | | |
| 3 | Urea | 1020 | mg | 340 | mg | 340 | mg | 340 | mg | 340 | mg |
| 4 | Formic acid | 0.005 | mg | 0.005 | mg | 0.0005 | mg | 0.0005 | mg | 0.0005 | mg |
| 5 | Ethanol | Q.s to 1 | ml | Qs to 1 | ml | Qs to 1 | ml | Qs to 1 | ml | Qs to 1 | ml |

### Manufacturing Process:

Eutectic mixture solvent is manufactured using choline chloride and Urea. Eutectic mixture solvent was fed into the product manufacturing vessel and decitabine was added to eutectic mixture solvent and dissolved. Formic acid was added to the above solution followed by addition of ethanol and stirred to get homogenous solution. The resulting decitabine solution was then filtered through a suitable sterilizing grade filter and filled into vials. The vial headspace was then blanketed with nitrogen to achieve a headspace oxygen content of less than 10 ppm, preferably less than 2 ppm. Finally, the vials were stoppered and sealed.

Decitabine formulation prepared according to the invention was tested for stability at 2° C.-8° C. and at 25° C./60% RH for a period of 6 months. Stability of examples 11, 12, 13, 14 and 15 are shown in Tables 11, 12, 13, 14 and 15 respectively.

TABLE 11
Stability data for product as obtained in Example - 11

| Condition/ | | 2° C.-8° C. | | | 25° C./60% RH | |
|---|---|---|---|---|---|---|
| Time Point | Initial | 1M | 3M | 6M | 1M | 3M |
| Moisture Content | 0.4 | 0.5 | 0.9 | 1.9 | 0.9 | 2.0 |
| (%) | | | | | | |
| Impurity - 1 (%) | ND | ND | ND | ND | 0.02 | 0.09 |
| Impurity -2 (%) | ND | 0.15 | 0.23 | 0.7 | 0.22 | 0.8 |
| Impurity -3 (%) | ND | ND | ND | ND | ND | ND |
| NFDU (%) | 0.42 | 0.42 | 0.62 | 0.89 | 0.56 | 1.2 |
| Total Impurities | 0.67 | 1.08 | 1.45 | 1.78 | 1.06 | 2.01 |
| (%) | | | | | | |
| Assay (%) | 99.9 | 99.3 | 98.5 | 98.4 | 98.9 | 98.1 |

*ND—Not detectable

TABLE 12
Stability data for product as obtained in Example - 12

| Condition/ | | 2° C.-8° C. | | | 25° C./60% RH | |
|---|---|---|---|---|---|---|
| Time Point | Initial | 1M | 3M | 6M | 1M | 3M |
| Moisture Content | 0.3 | 0.45 | 0.65 | 1.5 | 1.3 | 1.9 |
| (%) | | | | | | |
| Impurity - 1 (%) | ND | ND | ND | 0.05 | 0.09 | 0.14 |
| Impurity -2 (%) | ND | 0.29 | 0.32 | 0.56 | 0.32 | 0.7 |
| Impurity -3 (%) | ND | ND | ND | ND | ND | ND |
| NFDU (%) | 0.35 | 0.36 | 0.59 | 0.75 | 0.45 | 1.6 |
| Total Impurities | 0.45 | 1.2 | 1.42 | 1.65 | 1.23 | 1.95 |
| (%) | | | | | | |
| Assay (%) | 99.9 | 99.1 | 98.7 | 98.4 | 98.5 | 98.3 |

*ND—Not detectable

TABLE 13
Stability data for product as obtained in Example - 13

| Condition/ | | 2° C.-8° C. | | | 25° C./60% RH | |
|---|---|---|---|---|---|---|
| Time Point | Initial | 1M | 3M | 6M | 1M | 3M |
| Moisture Content | 0.5 | 0.65 | 0.9 | 1.6 | 1.3 | 1.9 |
| (%) | | | | | | |
| Impurity - 1 (%) | ND | ND | ND | ND | ND | ND |
| Impurity -2 (%) | ND | ND | 0.12 | 0.54 | 0.32 | 0.7 |
| Impurity -3 (%) | ND | ND | ND | ND | ND | ND |
| NFDU (%) | 0.52 | 0.65 | 0.85 | 1.2 | 0.45 | 1.6 |
| Total Impurities | 0.8 | 1.3 | 1.65 | 1.9 | 1.23 | 1.95 |
| (%) | | | | | | |
| Assay (%) | 99.2 | 99.1 | 98.7 | 98.1 | 98.5 | 98.3 |

*ND—Not detectable

TABLE 14
Stability data for product as obtained in Example - 14

| Condition/ | | 2° C.-8° C. | | | 25° C./60% RH | |
|---|---|---|---|---|---|---|
| Time Point | Initial | 1M | 3M | 6M | 1M | 3M |
| Moisture Content | 0.6 | 0.9 | 1.5 | 1.7 | 1.4 | 1.9 |
| (%) | | | | | | |
| Impurity - 1 (%) | ND | ND | ND | ND | 0.09 | 0.12 |
| Impurity -2 (%) | 0.02 | 0.12 | 0.29 | 0.56 | 0.7 | 0.78 |
| Impurity -3 (%) | ND | ND | ND | ND | ND | ND |
| NFDU (%) | 0.54 | 0.59 | 0.8 | 1.6 | 0.75 | 1.64 |
| Total Impurities | 0.7 | 1.3 | 1.6 | 1.8 | 1.5 | 1.8 |
| (%) | | | | | | |
| Assay (%) | 98.9 | 98.5 | 98.3 | 98.1 | 98.4 | 98.6 |

*ND—Not detectable

TABLE 15
Stability data for product as obtained in Example - 15

| Condition/ | | 2° C.-8° C. | | | 25° C./60% RH | |
|---|---|---|---|---|---|---|
| Time Point | Initial | 1M | 3M | 6M | 1M | 3M |
| Moisture Content | 0.5 | 0.7 | 1.3 | 1.9 | 1.8 | 2.1 |
| (%) | | | | | | |
| Impurity - 1 (%) | ND | ND | ND | ND | 0.07 | 0.12 |
| Impurity -2 (%) | ND | 0.12 | 0.54 | 0.69 | 0.5 | 0.8 |
| Impurity -3 (%) | ND | ND | ND | ND | ND | ND |
| NFDU (%) | 0.45 | 0.82 | 1.2 | 1.4 | 0.68 | 1.75 |
| Total Impurities | 0.8 | 1.2 | 1.6 | 1.95 | 1.23 | 2.05 |
| (%) | | | | | | |
| Assay (%) | 99.5 | 99.1 | 98.9 | 98.6 | 98.5 | 97.9 |

*ND—Not detectable

### Comparative Example 1

| S. No | Ingredients | Quantity (mg/mL) | |
|---|---|---|---|
| 1 | Decitabine | 10 | mg |
| 2 | Formic acid | 0.0005 | mg |
| 3 | Ethanol | Q.s to 1 | ml |

Manufacturing Process: Decitabine was dissolved in required quantity of ethanol and formic acid was added to it and stirred to get homogenous solution. The resulting decitabine solution was then filtered through a suitable sterilizing grade filter and filled into vials. The vial headspace was then blanketed with nitrogen to achieve a headspace oxygen content of less than 10 ppm, preferably less than 2 ppm. Finally, the vials were stoppered and sealed. Decitabine formulation prepared according to the comparative example was tested for stability at 2° C.-8° C. a for a period of 6 months as shown in Table-16.

TABLE 16
Stability data for product as obtained in Comparative Example 1.

| Condition/ | | 2° C.-8° C. | | |
|---|---|---|---|---|
| Time Point | Initial | 1M | 3M | 6M |
| Moisture Content (%) | 1.0 | 3.5 | 8.4 | 12.5 |
| Impurity - 1 (%) | ND | 0.01 | 0.12 | 0.09 |
| Impurity -2 (%) | ND | 0.52 | 1.5 | 5.2 |
| Impurity -3 (%) | ND | 0.06 | 0.09 | 0.23 |
| NFDU (%) | 0.35 | 2.7 | 5.8 | 9.2 |
| Total Impurities (%) | 0.7 | 3.12 | 9.5 | 17.0 |
| Assay (%) | 99.6 | 96.4 | 89.5 | 82.0 |

*ND—Not detectable

### Examples 16 to 18

Pharmaceutical formulation of decitabine prepared by static process with the ratio of decitabine bulk solution to molecular sieves as 1:0.5.

| | | Ex: 16 | | Ex: 17 | | Ex:18 | |
|---|---|---|---|---|---|---|---|
| | | Quantity | | Quantity | | Quantity | |
| S. No | Ingredients | (mg/mL) | | (mg/mL) | | (mg/mL) | |
| 1 | Decitabine | 10 | mg | 10 | mg | 10 | mg |
| 2 | Choline | 340 | mg | 340 | mg | 340 | mg |
| | Chloride | | | | | | |
| 3 | Urea | 170 | mg | 255 | mg | 340 | mg |
| 4 | Formic acid | 0.005 | mg | 0.0005 | mg | 0.0005 | mg |
| 5 | Ethanol | Qs to 1 | ml | Qs to 1 | ml | Qs to 1 | ml |

Manufacturing Process:
1. Eutectic mixture solvent is manufactured using choline chloride and Urea. Eutectic mixture solvent was fed into the product manufacturing vessel and decitabine was added to eutectic mixture solvent and dissolved. Formic acid was added to the above solution followed by addition of ethanol and stirred to get homogenous bulk solution.
2. Molecular sieved were added to the bulk solution of step 1 and incubated at 2°—8° C. for about 8 hours [weight ratio decitabine bulk solution to molecular sieves is 1:0.5 i.e 1 part of decitabine bulk solution and 0.5 parts of molecular sieves].
3. The bulk solution after 8 hours incubation was filtered through 0.2p filter and filled in vials and charged for stability at 2° C.-8° C. and at 25° C./60% RH for a period of 6 months. Stability of examples 16, 17, and 18 are shown in Tables 17, 18 and 19 respectively.

TABLE 17
Stability data for product as obtained in Example - 16

| Condition/ | | 2° C.-8° C. | | | 25° C./60% RH | | |
|---|---|---|---|---|---|---|---|
| Time Point | Initial | 1M | 3M | 6M | 1M | 3M | 6M |
| Moisture Content | ND | 0.1 | 0.2 | 0.3 | 0.25 | 0.4 | 0.5 |
| (%) | | | | | | | |
| Impurity - 1 (%) | ND | ND | ND | ND | ND | ND | ND |
| Impurity -2 (%) | ND | ND | ND | 0.03 | ND | 0.02 | 0.05 |
| Impurity -3 (%) | ND | ND | ND | ND | ND | ND | ND |
| NFDU (%) | 0.12 | 0.18 | 0.25 | 0.38 | 0.20 | 0.35 | 0.48 |
| Total Impurities | 0.2 | 0.25 | 0.38 | 0.45 | 0.3 | 0.5 | 0.7 |
| (%) | | | | | | | |
| Assay (%) | 100.4 | 100.2 | 99.7 | 99.6 | 99.7 | 99.5 | 99.2 |

*ND—Not detectable

TABLE 18
Stability data for product as obtained in Example - 17

| Condition/ | | 2° C.-8° C. | | | 25° C./60% RH | | |
|---|---|---|---|---|---|---|---|
| Time Point | Initial | 1M | 3M | 6M | 1M | 3M | 6M |
| Moisture Content | ND | ND | 0.1 | 0.2 | ND | 0.1 | 0.2 |
| (%) | | | | | | | |
| Impurity - 1 (%) | ND | ND | ND | ND | ND | ND | ND |
| Impurity -2 (%) | ND | ND | ND | ND | ND | ND | ND |
| Impurity -3 (%) | ND | ND | ND | ND | ND | ND | ND |
| NFDU (%) | 0.1 | 0.15 | 0.28 | 0.35 | 0.15 | 0.20 | 0.36 |
| Total Impurities | 0.18 | 0.2 | 0.35 | 0.4 | 0.25 | 0.3 | 0.5 |
| (%) | | | | | | | |
| Assay (%) | 100.5 | 100.1 | 99.9 | 99.7 | 100.2 | 99.9 | 99.6 |

*ND—Not detectable

TABLE 19
Stability data for product as obtained in Example - 18

| Condition/ | | 2° C.-8° C. | | | 25° C./60% RH | | |
|---|---|---|---|---|---|---|---|
| Time Point | Initial | 1M | 3M | 6M | 1M | 3M | 6M |
| Moisture Content | ND | 0.1 | 0.3 | 0.4 | 0.1 | 0.3 | 0.7 |
| (%) | | | | | | | |
| Impurity - 1 (%) | ND | ND | ND | ND | ND | ND | ND |
| Impurity -2 (%) | ND | ND | ND | ND | ND | ND | 0.02 |
| Impurity -3 (%) | ND | ND | ND | ND | ND | ND | ND |
| NFDU (%) | 0.1 | 0.19 | 0.35 | 0.45 | 0.19 | 0.4 | 0.6 |
| Total Impurities | 0.25 | 0.35 | 0.5 | 0.7 | 0.38 | 0.65 | 0.9 |
| (%) | | | | | | | |
| Assay (%) | 100.2 | 99.8 | 99.6 | 99.2 | 100.6 | 99.8 | 99.2 |

*ND—Not detectable

1. A stable liquid pharmaceutical composition of decitabine comprising decitabine and at least one pharmaceutically acceptable excipient, wherein moisture content of the liquid composition is less than about 2.0% by weight.

2. The stable liquid composition of claim 1, wherein moisture content of the liquid composition is less than 2.0% by weight when stored at 25° C./60% RH for at least 6 months.

3. The stable liquid composition of claim 1, wherein the total impurities of the composition is less than about 2.0% by weight when stored at 25° C./60% RH for at least 6 months.

4. The stable liquid composition of claim 1, wherein the alpha isomer impurity (Impurity-2) is less than about 1.0% by weight when stored at 25° C./60% RH for at least 6 months.

5. The stable liquid composition of claim 1, wherein the NFDU impurity and is less than about 1.0% by weight when stored at 25° C./60% RH for at least 6 months.

6. The stable liquid composition of claim 1, wherein the composition comprises pharmaceutically acceptable excipients selected from group consisting of eutectic solvents, at least one polar protic solvent, at least one polar aprotic solvent and an antioxidant.

7. The stable liquid composition of claim 6, wherein eutectic solvent consists halide salt and a hydrogen bond donor in the stoichiometric ratio of 0.5:3.0 to about 3.0 to about 0.5.

8. The stable liquid composition of claim 7, wherein halide salt is choline chloride.

9. The stable liquid composition of claim 8, wherein the hydrogen bond donor is selected from the group consisting of urea, Thiourea, 1-methyl urea, 1,1-dimethyl urea, 1,3-dimethyl urea, 2,3-xylenol, acetamide, benzamide, ethylene glycol, glycerol, and benzoic acid.

10. The stable liquid composition of claim 1, wherein composition is prepared by a process comprising a step of reducing the moisture content from the liquid compositions of decitabine, wherein the process comprises the steps of
a) preparing a bulk solution of decitabine comprising decitabine, eutectic solvent consisting of choline chloride and urea, a polar protic solvent and antioxidant,
b) incubating the bulk solution of decitabine of step a) with adsorbents or mixture of adsorbents in suitable ratio for suitable period of time, and
c) optionally filtering the decitabine solution from step b) by using suitable filter followed by filling into vials, stoppering and sealing.
